(19) United States
(12) Patent Application Publication
(10) Pub. No.: US 2008/0319473 A1
(43) Pub. Date: Dec. 25, 2008

(54) DEVICE FOR APPLYING PRESSURE TO A POCKET TO PREVENT HEMATOMA AFTER SURGICAL IMPLANTATION OF MEDICAL DEVICE AND METHOD OF USING THE SAME

(76) Inventor: Murray Rosenbaum, Boca Raton, FL (US)

Correspondence Address: MICHAEL RIES, 318 PARKER PLACE, OSWEGO, IL, 60543, US

(21) Appl. No.: 11/765,448

(22) Filed: Jun. 20, 2007

(51) Int. Cl. A61B 17/08

(52) U.S. Cl. 606/201

(57) ABSTRACT

A device for applying pressure to a pocket and for preventing hematoma after surgically implanting a medical device in the pocket. The device may have first and second portions for contacting the front and back sides of a patient, respectively, and a device for applying pressure to the pocket. The device may have cushions or padding for comfort. The pressure may be adjustable. The device allows a patient to ambulate with constant pressure on the pocket. The device may have a clamp, hinge, clasp, clip, spring, piston, pneumatic or inflatable device, band or any other device that applies pressure to the pocket.

### TECHNICAL FIELD AND BACKGROUND

The present invention generally relates to the field of surgical implantation of medical devices such as artificial pacemakers and implantable cardioverter defibrillators (ICDs) of all types. More specifically, the present invention relates to procedures involving the creation of a pocket created under the skin in the upper chest, where the pocket is adapted to receive the medical device, and a device for applying pressure to the pocket to prevent hematoma after implantation of the medical device. Also, the present invention relates to a method of using the device to apply pressure and prevent hematoma in such procedures.

The surgical implantation of artificial pacemakers and ICDs has become mainstream in medicine. Many times, these devices are implanted into patients who must remain on a blood thinner of some type. Examples of blood thinners include aspirin, clopidogrel (sold under the trade name PLAVIX, for example) or warfarin (sold under the trade name COUMADIN, for example). The risk of developing a serious hematoma after implantation of medical devices in a subdermal pocket is considerable and may be up to 10%. Conventional efforts have not effectively reduced the risk of developing a serious hematoma in and around a pocket after surgical implantation of artificial pacemakers and ICDs.

### BRIEF DESCRIPTION OF THE DRAWINGS

The present invention will be described by way of exemplary embodiments, but not limitations, illustrated in the accompanying drawings in which like references denote similar elements, and in which:

FIG. 1 is a side view of an embodiment of the present invention.

FIG. 2 is a back view of a left-sided embodiment of the present invention.

FIG. 3 is a back view of a right-sided embodiment of the present invention.

FIG. 4 is a front view of an embodiment of the present invention.

FIG. 5 is front view of a left-sided embodiment of the present invention on a human torso and placed over a pocket where a medical device has been implanted into a patient.

FIG. 6 is back view of the left-sided embodiment shown in FIG. 5.

FIG. 7 is front view of a right-sided embodiment of the present invention on a human torso and placed over a pocket where a medical device has been implanted into a patient.

FIG. 8 is back view of the left-sided embodiment shown in FIG. 7.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention, however, the order of description should not be construed as to imply that these operations are necessarily order dependent. In particular, these operations need not be performed in the order of presentation. The phrase in one embodiment is used repeatedly. The phrase generally does not refer to the same embodiment, however, it may. The terms comprising, having and including are synonymous, unless the context dictates otherwise.

The present inventors have sought to prevent hematoma formation by infusing clotting products into the subdermal pocket but have found this method to be generally ineffective. The present inventors have also sought to prevent hematoma formation by applying pressure over the pocket by having the patient lie in a prone position and applying sandbags, but this method is uncomfortable for the patient and the sandbags tend to slip off, for example, when the patient sits up. The present inventors have developed the present invention to overcome these problems.

The present inventors have significantly reduced the risk of developing a serious hematoma in and around a pocket after surgical implantation of artificial pacemakers and ICDs by using a device for applying pressure to the skin or clothing over and adjacent to the pocket. The device may be a clamp with padding on the inside of the clamp. The padding may be replaceable and enhances the comfort of the wearer of the clamp. The amount of pressure or tension applied by the clamp to the pocket may be adjustable. The shape of the clamp may be made so as to permit the movement of the patient's upper body including the patient's shoulders, arms and neck. As such, a patient is allowed to ambulate while maintaining constant pressure on the pocket, thus contributing to comfort while simultaneously preventing the risk of hematoma. In order to avoid the possibility of pressure sores, the portion of the clamp that makes contact with the back side of the patient may be made relatively larger than the portion of the clamp that makes contact with the front side of the patient, thus spreading the surface area of the pressure being applied through the clamp to the patient's body.

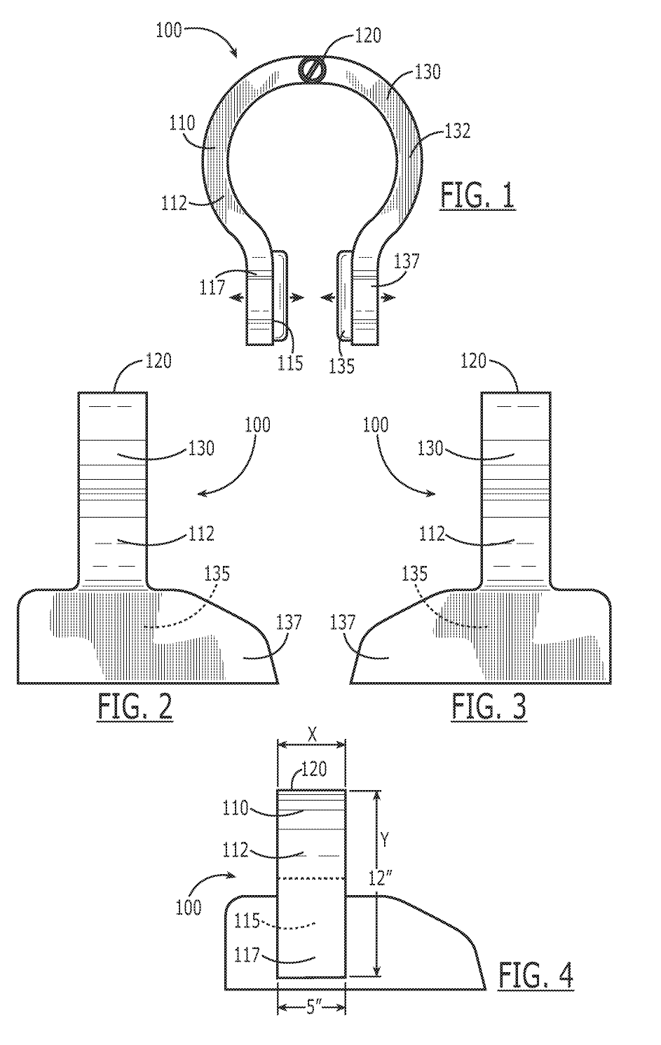

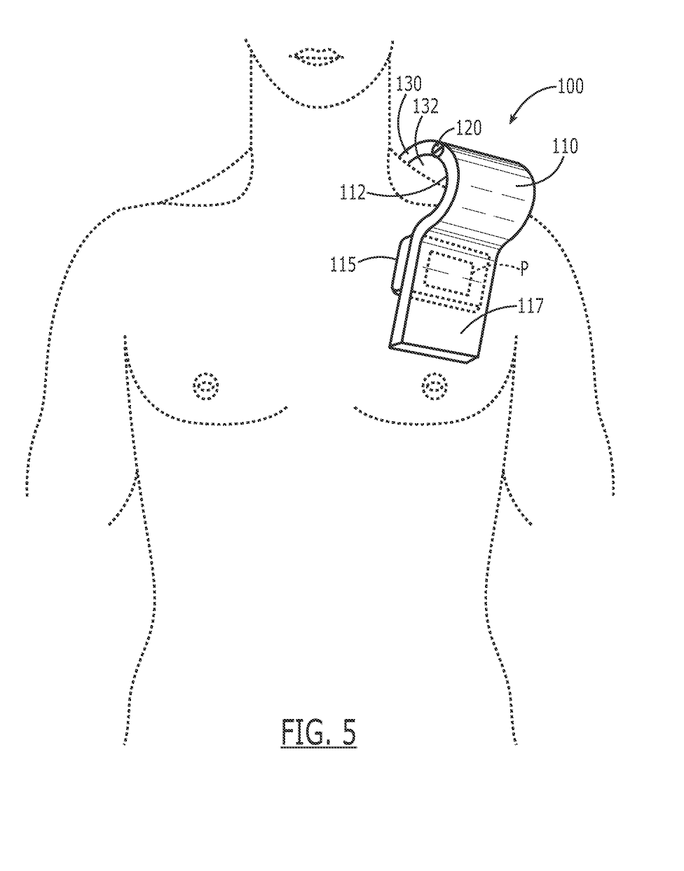

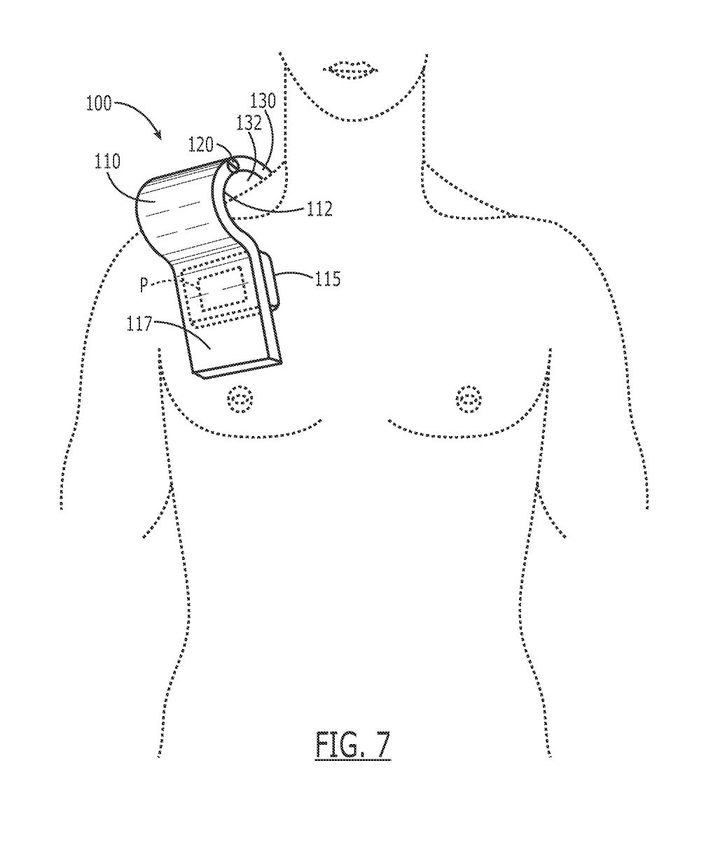

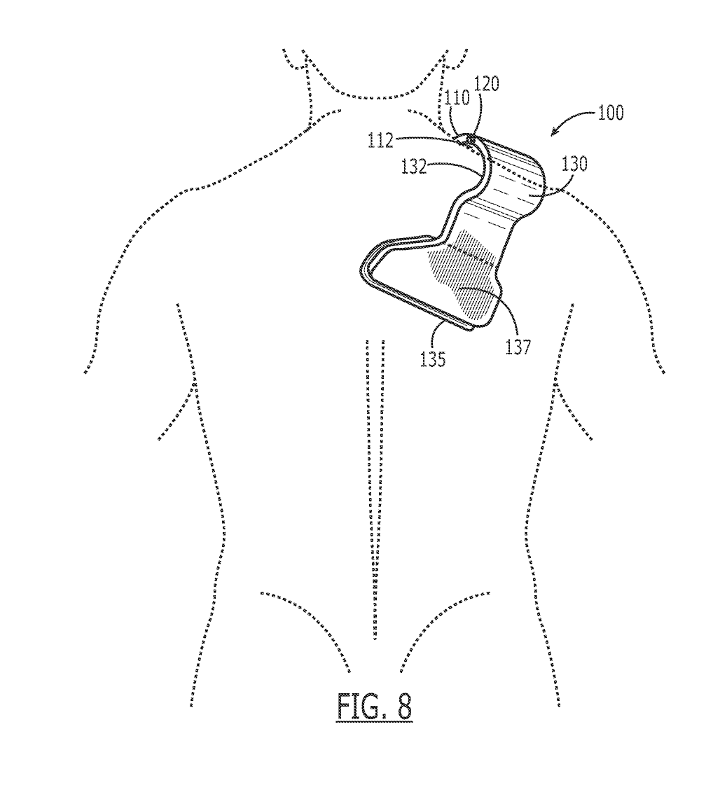

As shown, for example, in FIG. 1, the present invention is directed to a device 100 for applying pressure to a pocket P (see, for example, FIGS. 5 and 7) and for preventing hematoma after surgically implanting a medical device in the pocket P. The pocket P may be on the left side of the patient (FIG. 5) or the right side of the patient (FIG. 7). Although the pocket P is shown at a particular position on the chest of a patient, it is to be understood that the pocket P may be provided at any suitable position depending on the type of device being implanted. For artificial pacemakers and ICDs, the positions shown in FIGS. 5 and 7 are relatively common.

In one example, the device 100 may have overall dimensions as follows: a width X of about 5 inches and a height Y of about 12 inches when viewed from the front of the device 100, as shown in FIG. 4. However, any suitable dimensions may be used. For example, in pediatric applications, the dimensions would be generally smaller. The ratio of the width to the height need not necessarily be 5:12, and may be adjusted as necessary to accommodate the types of materials used to construct the device 100 and the amount of pressure being applied to the pocket. Also, the device 100 may be provided in multiples sizes to suit different size patients. That is, a larger patient will require a larger overall device 100 to accommodate his or her relatively thicker body. Alternately, the device 100 may be provided such that portions of the device 100 are allowed to extend and retract within a given range to accommodate various size patients. In other words, although the device 100 is shown in FIGS. 1 and 5-8 to have a two-piece construction, it is generally understood that the device may have a one-piece construction or may comprise additional pieces in order to perform the functions described herein.

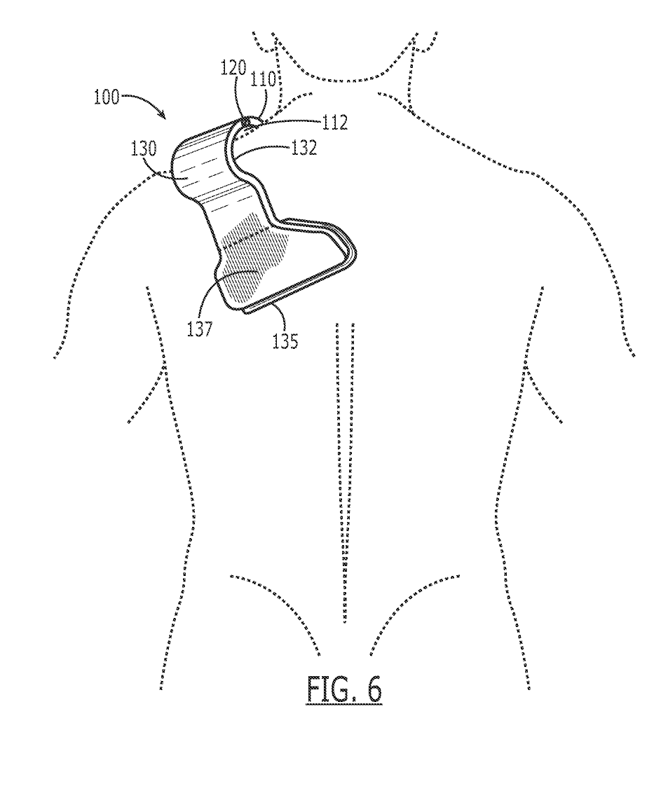

The device 100 may comprise a first portion 110 for contacting the front side of a patient (see, for example, FIG. 5 or FIG. 7). The device 100 may comprise a second portion 130 for contacting the back side of a patient (see, for example, FIG. 6 or FIG. 8). The device 100 is generally adapted to wrap around one side of a patient's body and over a portion of the body between the neck and one of the shoulders.

The first portion 110 and the second portion 130 may have an interior side and an exterior side. Cushioning may be attached to the interior side. The cushioning may be positioned to make contact with the skin or clothing of the patient (see, for example, FIG. 5 or FIG. 7). The first portion 110 and the second portion 130 may have an upper portion 112, 132 and a lower portion 117, 137, respectively, where the cushioning comprises a first cushion 115 (FIGS. 1, 5 and 7) and a second cushion 135 (FIGS. 1, 6 and 8), where the first cushion 115 is attached to the lower portion 117 of the first portion 110, and where the second cushion 135 is attached to the lower portion 137 of the second portion 130. The position of the cushions 115, 135 within the device 100 may be adjusted. For example, the cushions 115, 135 may be adapted to slide along the width or length of the first and second portions 110, 130, respectively, so that the cushions 115, 135 can be placed in an optimal position for comfort and for hematoma prevention. For example, the precise location of the pocket P will vary from patient to patient, and the present device 100 may be adapted to include an adjustable cushion 115 that can be positioned over the actual site of the pocket P after surgery. As shown in FIG. 5 or FIG. 7, the first cushion 115 may be positioned to make contact with the skin or clothing of the patient adjacent to and over the pocket P.

Although a pair of cushions 115,135 are shown and the cushions 115, 135 have a particular size relative to the overall dimensions of the device 100, it is to be understood that the entire interior surface of the device 100 may be lined with a cushioning material. Also, it is to be understood that any suitable size, thickness and material for the cushion may be used.

The first portion 110 and the second portion 130 may have an upper portion 112, 132 and a lower portion 117, 137, where the upper portion 112, 132 does not contact the skin or clothing of the patient (see, for example, FIGS. 5-8). Also, the first portion 110 and the second portion 130 may have an upper portion 112, 132 and a lower portion 117, 137, where the upper portion 112, 132 is adapted to permit movement of the upper body of a patient including the patient's shoulders, arms and neck. The upper portion 112,132 may be made to not contact the skin or clothing of the patient and to permit movement of the upper body of a patient including the patient's shoulders, arms and neck by providing the upper portion 112, 132 with a generally rounded or curved side cross-sectional shape, as shown best in FIG. 1. Although a rounded or curved shape is shown, it is to be understood that any suitable shape may be provided so long as it allows the upper portion 112, 132 of the device 100 to avoid contact with the skin or clothing of the patient and permit movement of the upper body of a patient including the patient's shoulders, arms and neck. The cross-sectional shape need not be perfectly round or circular and may be adapted to generally correspond with the shape of the portion of the human body that the device 100 is adapted to surround. In the case of over-the-shoulder use, the shape of this portion may correspond with the shoulder of a human patient, but be scaled up so that the skin or clothing of the patient is not touched by the device 100 and so that there is sufficient range of motion to permit the patient to sit up in bed, walk around, and, perhaps, permit the full range of motion of the upper body of a patient including the patient's shoulders, arms and neck.

The second portion 130 may be larger than the first portion 110. The present inventors have found that pressure sores may be avoided by providing the second portion 130 in a size that is larger than the first portion 110. Specifically, the portion of the device 100 that contacts the skin or clothing on the back side of the patient (FIGS. 6 and 8) should be larger than the portion of the device 100 that contacts the skin or clothing on the front side of the patient (FIGS. 5 and 7). The shape of the lower portion 137 of the second portion 130 may be generally L-shaped or boot-shaped with rounded corners, although any suitable shape or size may be provided.

The device 100 may comprise a means for applying pressure to the pocket P. The device 100 is not limited to any one means for applying pressure to the pocket P and is intended to include all suitable means for applying such pressure. For example, the device 100 may comprise one or more of the following: a clamp, a hinge, a clasp, a clip, a spring, a piston, a pneumatic or inflatable device, and a band.

For example, a spring clamp member 120 may be provided between the first portion 110 and the second portion 130. The member 120 may be inwardly biased, adjustable and hinged. The member 120 may have a bias toward a closed position. The member 120 may be adjusted to increase or decrease the pressure being applied to the pocket P. The member 120 may be a hinge coupled with a spring clamp in order to permit generally rotational movement between the first portion 110 and the second portion 130, where the axis of rotation is generally perpendicular to the page as shown in FIG. 1 and passes through the center of the member 120.

The member 120 may start out at a zero bias point where no pressure is applied to the device 100 or the pocket P and may be dialed up or pumped up to a higher pressure that is ideal for patient comfort and hematoma prevention and dialed down or pumped down when treatment is complete or when the device 100 needs to be repositioned or removed for some other reason. The device 100 may have a manual or digital gauge (not shown) for displaying the amount of pressure being applied through the device 100 and to the pocket P.

Although FIGS. 1-8 generally illustrate the present device 100 having first and second portions 110, 130 and a spring clamp member 120 between portions 110 and 130, it is generally understood that any suitable means for applying pressure to the pocket P may be used in lieu of the member 120 including, but not limited to one or more of a clamp, a hinge, a clasp, a clip, a spring, a piston, a pneumatic or inflatable device, and a band. The first and second portions 110, 130 may themselves be operable parts of the clamp, the hinge, the clasp, the clip, the spring, the piston, the pneumatic or inflatable device, or the band that is provided in order to provide pressure to the pocket P. Also, each of these terms is not intended to be limited to one type, but includes all types of devices fitting under each general classification.

Further, the present device 100 may be provided without a separate member 120, where the first and second portions 110, 130 may themselves serve as the means for applying pressure to the pocket P. For example, the first and second portions 110, 130 may be made of an elastic material having an inward bias.

The present invention is also directed to a method for applying pressure to a pocket P and for preventing hematoma after surgically implanting a medical device in the pocket P. After surgery, a practitioner, such as a doctor or nurse, attaches the device 100 of the present invention to the patient. The device 100 may comprise one or more of the features described in detail above.

While the present invention has been related in terms of the foregoing embodiments, those skilled in the art will recognize that the invention is not limited to the embodiments depicted. The present invention can be practiced with modification and alteration within the spirit and scope of the appended claims. Thus, the description is to be regarded as illustrative instead of restrictive on the present invention.

What is claimed is:

1. A device for applying pressure to a pocket and for preventing hematoma after surgically implanting a medical device in the pocket, the device comprising:
a first portion for contacting the front side of a patient;
a second portion for contacting the back side of a patient; and
a means for applying pressure to the pocket.

2. The device of claim 1, wherein the first portion and the second portion have an interior side and an exterior side, and wherein cushioning is attached to the interior side.

3. The device of claim 2, wherein the cushioning is positioned to make contact with the patient.

4. The device of claim 2, wherein the first portion and the second portion have an upper portion and a lower portion, wherein the cushioning comprises a first cushion and a second cushion, wherein the first cushion is attached to the lower portion of the first portion, and wherein the second cushion is attached to the lower portion of the second portion.

5. The device of claim 4, wherein the first cushion is positioned to make contact with the patient adjacent to and over the pocket.

6. The device of claim 1, wherein the first portion and the second portion have an upper portion and a lower portion, and wherein the upper portion does not contact the patient.

7. The device of claim 1, wherein the first portion and the second portion have an upper portion and a lower portion, wherein the upper portion is adapted to permit movement of the upper body of a patient.

8. The device of claim 7, wherein the upper portion has a generally curved side cross-sectional shape.

9. The device of claim 1, wherein the second portion is larger than the first portion.

10. The device of claim 1, wherein the device is a selected one of a clamp, a hinge, a clasp, a clip, a spring, a piston, a pneumatic device, an inflatable device, and a band.

11. A method for applying pressure to a pocket and for preventing hematoma after surgically comprising:
implanting a medical device in the pocket;
contacting a front side of a patient with a first portion;
contacting a back side of a patient with a second portion; and
applying pressure to the pocket.

12. The method of claim 11, wherein the first portion and the second portion have an interior side and an exterior side, and wherein cushioning is attached to the interior side.

13. The method of claim 12, wherein the cushioning is positioned to make contact with the patient.

14. The method of claim 12, wherein the first portion and the second portion have an upper portion and a lower portion, wherein the cushioning comprises a first cushion and a second cushion, wherein the first cushion is attached to the lower portion of the first portion, and wherein the second cushion is attached to the lower portion of the second portion.

15. The method of claim 14, wherein the first cushion is positioned to make contact with the patient adjacent to and over the pocket.

16. The method of claim 11, wherein the first portion and the second portion have an upper portion and a lower portion, and wherein the upper portion does not contact the patient.

17. The method of claim 11, wherein the first portion and the second portion have an upper portion and a lower portion, wherein the upper portion is adapted to permit movement of the upper body of a patient.

18. The method of claim 17, wherein the upper portion has a generally curved side cross-sectional shape.

19. The method of claim 11, wherein the second portion is larger than the first portion.

20. The method of claim 11, wherein the device is a selected one of a clamp, a hinge, a clasp, a clip, a spring, a piston, a pneumatic device, an inflatable device, and a band.
